SEQUENCE LISTING (6 sequences)

SEQ ID NO: 1 - protein, 852 aa, Homo sapiens
MLITERKHFRSGRIAQSMSEANLIDMEAGKLSKSCNITECQDPDLLHNWPDAFTLRGNNASKVANPFWNQLSASNPFLDDITQLRNNRKRNNISILKEDPFLFCREIENGNSFDSSGDELDVHQLLRQTSSRNSGRSKSVSELLDILDDTAHAHQSIHNSDQILLHDLEWLKNDREAYKMAWLSQRQLARSCLDLNTISQSPGWAQTQLAEVTIACKVNHQGGSVQLPESDITVHVPQGHVAVGEFQEVSLRAFLDPPHMLNHDLSCTVSPLLEIMLGNLNTMEALLLEMKIGAEVRKDPFSQVMTEMVCLHSLGKEGPFKVLSNCYIYKDTIQVKLIDLSQVMYLVVAAQAKALPSPAATIWDYIHKTTSIGIYGPKYIHPSFTVVLTVCGHNYMPGQLTISDIKKGGKNISPVVFQLWGKQSFLLDKPQDLSISIFSCDPDFEVKTEGERKEIKQKQLEAGEVVHQQFLFSLVEHREMHLFDFCVQVEPPNGEPVAQFSITTPDPTPNLKRLSNLPGYLQKKEEIKSAPLSPKILVKYPTFQDKTLNFSNYGVTLKAVLRQSKIDYFLEYFKGDTIALLGEGKVKAIGQSKVKEWYVGVLRGKIGLVHCKNVKVISKEQVMFMSDSVFTTRNLLEQIVLPLKKLTYIYSVVLTLVSEKVYDWKVLADVLGYSHLSLEDFDQIQADKESEKVSYVIKKLKEDCHTERNTRKFLYELIVALLKMDCQELVARLIQEAAVLTSAVKLGKGWRELAEKLVRLTKQQMEAYEIPHRGNTGDVAVEMMWKPAYDFLYTWSAHYGNNYRDVLQDLQSALDRMKNPVTKHWRELTGVLILVNSLEVLRVTAFSTSEEV

SEQ ID NO: 2 - DNA, 2559 bp, Homo sapiens
atgctaatcactgaaagaaaacattttcggtcaggaagaattgcacaaagtatgtctgaagcaaatttgattgacatggaagctggaaaactctcaaaaagttgcaatattacagaatgccaggacccagacttgcttcacaattggccggatgctttcacccttcgtggtaataatgcttccaaagttgcaaatccattctggaatcaactgtctgcttctaacccatttttggatgacataactcaactaagaaataacaggaagagaaataatatttccatcttaaaggaagatccttttcttttctgtagagaaatagaaaatggaaattcttttgattcctccggtgatgaacttgatgtgcatcagttacttaggcagacttcctcaagaaattctggaagatctaaaagtgtttcagaacttctggacattttagacgacacagcacatgcccatcagagtatacataactctgaccagatcctactacacgacttagagtggcttaaaaatgatcgggaggcttataaaatggcttggttaagtcaacgccagctggcccgctcctgccttgatttgaatacaattagtcagagccctggatgggcccagacacaacttgcggaggtcaccatagcttgcaaagtaaaccatcaaggagggtcagtacaattacctgaatcagacatcactgttcatgtgccccaaggtcatgtggctgtgggagaattccaagaggtgtctctaagggctttccttgatccgccacacatgcttaaccatgatctttcgtgcactgtgagcccgttgttggaaatcatgttaggcaacctcaatacaatggaagcccttttgctggagatgaaaattggggctgaagtaagaaaggatcctttcagccaagtcatgacagaaatggtgtgtttacacagcttgggtaaagaaggcccttttaaagttttaagcaactgctacatttataaagacaccatccaagtcaagctaatcgacttgagtcaggtaatgtatctagtggttgctgcacaagctaaagctcttccgtcaccagctgccaccatttgggattatatccacaaaaccacctcaattggaatttatggacccaaatatatccatcccagttttactgttgttttaacagtttgtggacacaattatatgccaggacagcttacaatttctgatattaagaagggtggaaaaaacatatctccagttgtgtttcagctctgggggaagcagtcatttttacttgacaagccacaagatttaagtatttctattttttcctgtgatcctgattttgaagtaaagacagaaggagaaaggaaagaaattaaacaaaagcagttggaagcaggtgaagtagttcatcaacaatttttattttctttagttgagcacagagagatgcacttgtttgatttttgtgttcaagtggagcctcccaatggtgaaccagttgcacagttctctatcactactcctgatccaaccccaaacctaaaaagactctcgaatctgccaggctatttgcagaagaaggaggaaatcaagtctgctcctttatcaccaaaaattcttgttaaatatcctacatttcaagataaaacattgaactttagcaactatggggtaaccctgaaggcagtgctaagacaaagcaagattgattacttccttgaatatttcaaaggggacacaatagctctcctcggggaaggtaaggtaaaagctattggtcagtccaaagtgaaagaatggtatgtaggagtcctcagaggtaagattggacttgtacactgcaaaaatgtcaaggtgatttcaaaggagcaagtaatgtttatgtcagatagtgtctttacaaccagaaatcttcttgaacagattgtcctgcctttaaaaaaattgacttatatctactcagttgtattaaccttggtgtcagaaaaagtttatgattggaaagttttagctgatgtcctgggttactcacatctgtccctggaagattttgatcaaattcaagcagacaaagaatcagagaaagtttcttatgttataaagaagttaaaggaagattgccacacagagagaaatacaaggaagtttctgtatgaacttattgtggctcttctgaaaatggattgccaagagttagtcgcacgtctcatccaagaagctgctgttctgacttcagctgtcaagcttggaaaaggctggagggaactagctgaaaagttagtacgactcacaaagcaacaaatggaggcatatgaaattcctcatcgaggaaacactggagatgttgctgttgagatgatgtggaaacctgcctatgattttctgtatacctggagtgctcactatggaaataactacagagatgtgttacaagaccttcagtcagctttggacagaatgaaaaaccctgtgactaaacactggagagaattaactggagttttaatactagtaaattctttggaggttttgagagtaactgcattctccacttctgaggaagtatag

SEQ ID NO: 3 - DNA, 21 bp, ARTIFICIAL SEQUENCE
  SYNTHETIC CONSTRUCT
ttcttttgattcctccggtga

SEQ ID NO: 4 - DNA, 20 bp, ARTIFICIAL SEQUENCE
  SYNTHETIC CONSTRUCT
actctgatgggcatgtgctg

SEQ ID NO: 5 - DNA, 32 bp, ARTIFICIAL SEQUENCE
  SYNTHETIC CONSTRUCT
gcagacttcctcaagaaattctggaagatcta

SEQ ID NO: 6 - DNA, 32 bp, ARTIFICIAL SEQUENCE
  SYNTHETIC CONSTRUCT
agtgtttcagaacttctggacattttagacga

(19) United States
(12) Patent Application Publication
(10) Pub. No.: US 2014/0356868 A1
(43) Pub. Date: Dec. 4, 2014

(54) MACC1 AS A PROGNOSTIC BIOMARKER FOR HEPATOBILIARY TUMORS

(71) Applicant: Charite - Universitaetsmedizin Berlin, Berlin (DE)

(72) Inventors: Ulrike STEIN, Panketal (DE); Peter M. SCHLAG, Berlin (DE); Andri LEDERER, Berlin (DE); Daniel SEEHOFER, Berlin (DE); Pia HERMANN, Berlin (DE)

(21) Appl. No.: 14/291,831

(22) Filed: May 30, 2014

Related U.S. Application Data
(60) Provisional application No. 61/829,669, filed on May 31, 2013.

(51) Int. Cl. C12Q 1/68; C07K 16/22; G01N 33/574

(52) U.S. Cl. CPC C12Q 1/6886; G01N 33/57438; C07K 16/22; C12Q 2600/158; C12Q 2600/118; G01N 2333/475
USPC 435/6.11; 435/6.14; 435/6.12; 436/501; 435/7.92; 436/86; 536/24.31; 530/387.9

(57) ABSTRACT

The present invention is directed to a method of diagnosis and prognostication of a hepatobiliary tumor, the method comprising the step of determining expression of MACC1 polypeptide or of a nucleic acid encoding said MACC1 polypeptide in a biological sample.

### CROSS-REFERENCE TO RELATED APPLICATION

This application claims the benefit of Provisional Application Ser. No. 61/829,669, filed May 31, 2013, which is hereby incorporated by reference in its entirety, for all purposes, herein.

### FIELD OF THE INVENTION

The present invention refers to a new method of diagnosing and prognostication of hepatobiliary tumors, wherein the method is based on the use of MACC1 as a biomarker.

### DESCRIPTION

The most common malignant hepatobiliary tumors are hepatocellular carcinoma (HCC) and cholangiocarcinomas, which can be divided in hilar cholangiocarcinomas (Klatskin tumors) and intrahepatic cholangiocarcinoms (ICC). The latter are rare diseases, with an incidence rate<1/100.000 in Europe and in the USA. However they are the second most common primary tumor of the liver and account for approximately 20% of all deaths related to liver malignancies. Hepatocellular carcinoma (HCC) is the most common primary liver tumor in adults, the fifth most common malignancy overall, and the third most common cause of cancer related death worldwide.

Besides surgical excision, the current therapeutic options for Klatskin tumors, ICC, and HCC are very limited, and most patients have advanced disease at presentation and are rendered inoperable or relapse shortly after surgery. Neoadjuvant and adjuvant radio-chemotherapy is mainly carried out in clinical trials, and treatment outcome is still not satisfying. Tumor free survival strongly depends on early diagnosis, complete tumor resection (tumor free resection borders RO) and absence of lymph node and distant metastasis. Patients fulfilling the Milan criteria can be approved for liver transplantation, which yields a 5 year survival rate of >50% in HCC. However, this treatment option is highly limited by the lack of available donor organs.

Even though the recurrence rate for Klatskin tumors, ICC, and HCC after surgery is high, it is very heterogenic, and some patients could highly benefit of a curative surgical approach. Thus, it is of utmost interest to identify these patients when doing the cancer staging. Biomarkers that identify patients at high risk of tumor recurrence and metastasis could be an essential tool to guide the treatment of patients with hepatobiliary tumors.

In WO 2005/010042 A1 our group disclosed a new regulator of the HGF/Met/MAPK pathway, called Metastasis-associated in colon cancer 1 (MACC1; also called 7a5/Prognostin). MACC1 was identified by a genome-wide search for differentially expressed genes in human colon cancer tissue, metastases, and normal tissue. The MACC1 gene is located on human chromosome 7 (7p21.1), the same chromosome that contains the genes of Met (7q31.2) and HGF (7q21.1). MACC1 turned out to be a powerful biomarker for the prediction of metachronous distant metastasis as well as survival in colon cancer patients. Based on the expression level of MACC1 mRNA, the negative and positive prediction of distant metastasis was 80% and 74%, respectively.

There remains a need for further biomarkers with prognostic significance with respect to hepatobiliary tumors.

In a first aspect, the present invention is directed to a method of diagnosis of a hepatobiliary tumor, the method comprising the step of determining the expression level of MACC1 polypeptide or of a nucleic acid encoding said MACC1 polypeptide in a biological sample, wherein said MACC1 polypeptide has at least 80% identity to the amino acid sequence with SEQ ID NO. 1.

It has surprisingly been found that expression of MACC1 is significantly elevated in hepatobiliary tumors, in particular in cholangiocarcinomas (Klatskin tumors and ICC). Thus, MACC1 is particularly suitable as biomarker in a method of diagnosis of hepatobiliary tumors. Elevated expression of MACC1 is significantly correlated not only with presence of hepatobiliary tumors but also correlates significantly with increased tumor infiltration, advanced stage of disease, poor prognosis, poor overall survival, poor disease-free survival and increased likelihood of occurrence of tumor recurrences.

The method of the invention is directed to diagnosis of hepatobiliary tumors. Hepatobiliary tumors comprise all malignant tumorous diseases which develop from hepatocytes, intrahepatic bile duct, extrahepatic bile duct and gall bladder. In particular, hepatocellular carcinoma (HCC), gall bladder carcinoma and cholangiocarcinoma represent hepatobiliary tumors, wherein cholangiocarcinomas can be divided in intrahepatic cholangiocarcinomas (ICC) and hilar cholangiocarcinomas, also called Klatskin tumors. In the method of the invention, the hepotabiliary tumor to be diagnosed is preferably a cholangiocarcinoma or a gall bladder carcinoma, more preferably the hepatobiliary tumor is a cholangiocarcinoma, even more preferably the hepatobiliary tumor is a Klatskin tumor.

The method of the invention is directed to a method of diagnosis of a hepatobiliary tumor. The term “diagnosis” as used herein does not only refer to detection of presence or absence of tumorous tissue or disease. It also encompasses more specific meanings like overall or specific prognosis, determination of severity of the disease, tumor staging, determination of tumor infiltration stage, determination of stage of disease, prognosis on overall survival, prognosis on disease-free survival and prognosis on likelihood of occurrence of tumor recurrences. The result of the method of diagnosis of the invention may be used as basis for optimization of disease management, for further decisions like exclusion of certain diseases, decisions on further treatment options like decisions on enrolment of particular patients for tumor surgery e.g. for complete or partial tumor resection and liver transplantation.

It has been found that elevated expression level of MACC1 in a biological sample is indicative for presence of hepatobiliary tumorous tissue in said biological sample. Thus, the method of the invention allows for use in diagnosis of hepatobiliary tumors like e.g. Klatskin tumors.

It has been found that elevated expression level of MACC1 in a biological sample is indicative for advanced stage of infiltration of hepatobiliary tumor. Thus, the method of the invention allows for use in staging of hepatobiliary tumors like e.g. Klatskin tumors.

It has been found that elevated expression level of MACC1 in a biological sample is indicative of increased likelihood of tumor recurrence. Thus, the method of the invention allows for use in prognosis on development of recurrences of hepatobiliary tumors like e.g. Klatskin tumors.

It has been found that elevated expression level of MACC1 in a biological sample is indicative of poor likelihood of prolonged survival. Thus, the method of the invention allows for use in overall prognosis of disease progression and, in particular, for prognosis on overall survival and disease-free survival.

The method of the invention comprises the step of determining the expression level of MACC1 polypeptide or of a nucleic acid encoding said MACC1 polypeptide. MACC1 polypeptide and nucleic acids encoding said polypeptide are known to the person skilled in the art. MACC1 polypeptide sequence and the sequence of a nucleic acid molecule encoding for said polypeptide have been disclosed in WO 2005/010042 A1, the content of which is incorporated herein by reference in its entirety.

Preferably, the MACC1 polypeptide as used for the purpose of the present invention has at least 80%, 90%, 95%, 99% or 100% identity to the amino acid sequence with SEQ ID NO. 1.

For the purpose of the present invention, sequence “identity” can be objectively determined by any of a number of methods. The skilled person is well aware of these methods and can choose a suitable method without undue burden. A variety of methods for determining relationships between two or more sequences (e.g. identity, similarity and/or homology) are available and well known in the art. The methods include manual alignment, computer assisted sequence alignment and combinations thereof, for example. A number of algorithms (which are generally computer implemented) for performing sequence alignment are widely available or can be produced by one of skill. The degree of identity of one amino acid sequence or nucleotide sequence to another can be determined by following the algorithm BLAST by Karlin and Altschul (Proc. Natl. Acad. Sci. USA 90: 5873-5877, 1993). Programs such as BLASTN and BLASTX developed based on this algorithm (Altschul et al. (1990) J. Mol. Biol. 215: 403-410) may be used. To analyze a nucleotide sequence according to BLASTN based on BLAST, the parameters are set, for example, as score=100 and word length=12. On the other hand, parameters used for the analysis of amino acid sequences by the BLASTX based on BLAST include, for example, score=50 and word length=3. Default parameters of each program are used when using BLAST and Gapped BLAST program. Specific techniques for such analysis are known in the art.

A nucleic acid sequence is said to “encode” a polypeptide, if the nucleic acid can be transcribed (in spliced or unspliced form) and/or translated into said polypeptide. The skilled person is well aware of the degeneracy of the genetic code, allowing for a number of different nucleic acid sequences to encode for the same amino acid sequence or polypeptide, and has no difficulty in determining whether a given nucleic acid encodes for a given amino acid sequence or polypeptide.

In the method of the invention, the expression level of MACC1 polypeptide or of a nucleic acid encoding said MACC1 polypeptide is determined. The term “expression level” is used in its art recognized meaning and refers to the amount of a gene product being present at a given point in time. A gene product in this sense can be a nucleic acid based transcript of said gene like e.g. an unspliced, partially spliced or fully spliced mRNA (transcript) or a polypeptide based translation product of said gene. “Expression of a gene” or “expression of a nucleic acid” usually refers to transcription of genomic DNA into RNA (optionally including modification of the RNA, e.g. splicing), translation of RNA into a polypeptide (possibly including subsequent modification of the polypeptide, e.g. posttranslational modification), or both transcription and translation, as indicated by the context.

Usually the expression level of a gene is determined either on the level of translated polypeptide or protein or on the level of transcribed nucleic acid like e.g. of fully or partially spliced mRNA. If the expression level of MACC1 polypeptide is determined, the amount of translated MACC1 polypeptide present in the biological sample or in a part thereof is determined directly or indirectly in absolute or relative terms. If the expression level of a nucleic acid encoding for MACC1 polypeptide is determined, the amount of transcribed nucleic acid like e.g. mRNA encoding for MACC1 polypeptide present in the biological sample or in a part thereof is determined directly or indirectly in absolute or relative terms.

The method of the invention is not limited to a particular method or technique to determine expression level of MACC1 polypeptide or of a nucleic acid encoding said MACC1 polypeptide. The skilled person is well aware of a number of different techniques which allow for successful and reliable determination of expression levels of proteins, polypeptides and nucleic acid molecules in a given sample. Preferably, techniques are used which allow for direct or indirect determination of expression levels either in absolute terms or in relative terms. Techniques which are known to be particularly useful in determining expression level of MACC1 polypeptide or of a nucleic acid encoding said MACC1 polypeptide comprise techniques based on hybridisation, PCR, quantitative or semi-quantitative RT-PCR, “real-time” (RT)-PCR, antigen-antibody binding, ELISA, optical proteome analysis, one- or multi-dimensional gel electrophoresis, analysis by mass spectrometry, chromatography, sequencing procedure, methylation analysis, SNP-determination or on a combination including one or more of these methods.

In the method of the invention, the expression level of MACC1 in a biological sample is determined. A biological sample may comprise, be derived from or consist of biological material like e.g. cells, tissue or fluid derived from a biological source or of constituents which have been derived therefrom. Preferably, the biological sample is based on biological material derived from a subject. Said subject can be an animal, preferably it is a mammal, more preferably said subject is a human, even more preferably said subject is a human patient in need of a diagnosis of hepatobiliary tumors. The biological sample may be derived from a biopsy of a tissue to be investigated, preferably from a biopsy comprising hepatic, biliary and/or putatively tumorous tissue. The biological sample may also be derived from a biliary brush cytology or by an other technique to collect the aforementioned type of tissue. The biological sample may be derived from a subject to be investigated, preferably from a patient who is in need of such a diagnosis.

The method of the invention can be configured to be an in vitro method. In this case, the step of isolating the biological probe from a subject does not form a constitutive part of the method.

Alternatively, the method of the invention may be configured to comprise the additional step of isolating the biological sample to be investigated from a subject.

The method of the invention is particularly informative if it is determined whether the expression level of MACC1 polypeptide or of a nucleic acid encoding MACC1 polypeptide is elevated.

The expression level of MACC1 polypeptide or of a nucleic acid encoding said MACC1 polypeptide is elevated if the expression level in the biological sample exceeds the expression level in a comparative sample and/or if the expression level in the biological sample exceeds a predetermined threshold value. If elevated expression of MACC1 is determined by comparison to expression level in a comparative sample, said comparative sample may be a standard sample comprising a known amount of MACC1 polypeptide or nucleic acid encoding MACC1. Such a comparative standard sample may be derived from a biological sample of the same type which is disease-free. In this case the comparative biological sample may be derived from the same subject or from another subject of the same or of a closely related species. Alternatively, the comparative sample may be a sample wherein a known amount of MACC1 polypeptide or of a nucleic acid encoding MACC1 or a part thereof has been spiked in.

Alternatively, the expression level of MACC1 polypeptide or of a nucleic acid encoding said MACC1 polypeptide is elevated if the expression level in the biological sample exceeds a predetermined threshold value. In this case, the threshold value and the expression level of MACC1 in a biological sample may be given as an absolute value. Such an absolute value may be dimensionless or may be given as total number of copies per unit, wherein the unit may be a volume unit, a weight unit or the like. Alternatively the threshold value and the expression level of MACC1 in the biological sample may be given as a relative expression value. In this case the relative expression value is based on MACC1 expression related to expression of a certain reference gene or protein, like a common housekeeping gene which usually does not vary substantially in its expression level, or to a calibrator which has been spiked into the biological sample.

The method of the invention may comprise the following steps:
determining the expression level of MACC1 polypeptide or of a nucleic acid encoding said MACC1 polypeptide in a biological sample;
determining the expression level of MACC1 polypeptide or of a nucleic acid encoding said MACC1 polypeptide in a comparative sample; and
comparing the expression level of the biological sample with the expression level of the comparative sample.

Alternatively, the method of the invention may comprise the steps:
determining the expression level of MACC1 polypeptide or of a nucleic acid encoding said MACC1 polypeptide in a biological sample; and
comparing the expression level of the biological sample with a predetermined threshold value.

In the method of the invention, the expression level of MACC1 polypeptide can be determined using a polypeptide which binds specifically to the MACC1 polypeptide, wherein said MACC1 polypeptide has at least 80% identity to the amino acid sequence with SEQ ID NO. 1. A number of different types of polypeptides can be used to arrive at specific binding to MACC1 polypeptide. The use of antibodies being specific for MACC1 polypeptide appears to be particularly suitable. Such antibodies are either polyclonal or monoclonal; preferably a monoclonal antibody is used. According to the present invention, the term antibody can also subsume genetically produced and potentially modified antibodies or antigen-binding parts thereof, like e.g. chimeric antibodies, humanised antibodies, multifunctional antibodies, bi- or oligospecific antibodies, single-stranded antibodies, F(ab)- or F(ab)2-fragments (see e.g. EP-B1-0 368 684, U.S. Pat. No. 4,816,567, U.S. Pat. No. 4,816,397, WO 88/01649, WO 93/06213, WO 98/24884). These references are incorporated by reference herein in their entireties.

In the method of the invention, the expression level of a nucleic acid encoding for MACC1 polypeptide is determined using a nucleic acid molecule that hybridizes under stringent conditions to a nucleic acid molecule encoding MACC1 polypeptide or to the reverse complement thereof, wherein said MACC1 polypeptide has at least 80% identity to the amino acid sequence with SEQ ID NO. 1. Preferably, a nucleic acid molecule is used which hybridizes under stringent conditions to a nucleic acid sequence with SEQ ID NO. 2 or to a reverse complement thereof. The skilled person is well aware of methods and techniques of how to generate suitable nucleic acid molecules and how to perform hybridisation reactions under stringent conditions. Stringent hybridization conditions of the present invention include conditions such as: 6 M urea, 0.4% SDS, and 0.5×SSC; and those which yield a similar stringency to these conditions. Hybridisation may be performed under conditions with even higher stringency, for example, 6 M urea, 0.4% SDS, and 0.1×SSC.

The present invention is also directed to an isolated nucleic acid molecule for use in a method of diagnosis of a hepatobiliary tumor according to the present invention, wherein said method comprises the step of isolating the biological sample from a subject, characterized in that said nucleic acid molecule hybridizes under stringent conditions to a nucleic acid sequence encoding MACC1 polypeptide or to the reverse complement thereof, wherein said MACC1 polypeptide has at least 80% identity to the amino acid sequence with SEQ ID NO. 1. Preferably, said nucleic acid molecule hybridizes under stringent conditions to a nucleic acid sequence with SEQ ID NO. 2 or to a reverse complement thereof.

In a further aspect, the present invention is directed to an isolated polypeptide for use in a method of diagnosis of a hepatobiliary tumor according to the present invention, wherein said method comprises the step of isolating the biological sample from a subject, wherein said isolated peptide is characterized in that it binds specifically to the MACC1 polypeptide, wherein said MACC1 polypeptide has at least 80% identity to the amino acid sequence with SEQ ID NO. 1. Preferably, said isolated polypeptide is an antibody as specified above.

In a further aspect, the present invention is directed to the use of MACC1 polypeptide or of a nucleic acid encoding said MACC1 polypeptide, wherein said MACC1 polypeptide has at least 80% identity to the amino acid sequence with SEQ ID NO. 1, as a biomarker in the diagnosis if hepatobiliary tumors, preferably of cholangiocarcinoma and gall bladder carcinoma, more preferably of Klatskin tumors.

The present invention is also directed to the use of a method of the invention for evaluating the effectiveness of a treatment of a hepatobiliary tumor.

In a further aspect, the present invention is also directed to a method for identifying a candidate compound useful in treatment of hepatobiliary tumors, said method comprising the steps of:
contacting a mammal subject presenting a hepatobiliary tumor with a candidate compound:
determining the expression level of MACC1 polypeptide or of a nucleic acid encoding said MACC1 polypeptide in a biological sample isolated from said subject;
   wherein a decrease in the expression level indicates that the candidate compound is useful in treatment of hepatobiliary tumors.

The invention will now be further described by way of particular examples.

### FIGURES

FIG. 1 MACC1 mRNA expression in Klatskin tumors (a) and intrahepatic cholangiocarcinoma (b) with corresponding peritumorous normal liver tissue, determined by quantitative real-time RT-PCR. MACC1 expression is significantly higher in tumor tissue of Klatksin tumors (n=76) and intrahepatic cholangiocarcinoma (n=80) than in the corresponding normal tissue. (Wilcoxon signed-rank test)

FIG. 2 MACC1 mRNA expression in Klatskin tumors with a size of pT1 or pT2 (median 4.14; n=16) and pT3 or pT4 (median 8.03; n=54), measured by quantitative real-time RT-PCR. MACC1 mRNA expression is significantly higher in patients with a tumor size of pT3 and pT4 than with pT1 and pT2. (Mann Whitney U Test)

FIG. 3 MACC1 mRNA expression in Klatskin tumor tissue of UICC stages I+II (n=63) and stages III+IV (n=5) as determined by quantitative real-time RT-PCR. MACC1 mRNA expression does not differ significantly between the different UICC stages. (Mann Whitney U Test)

FIG. 4 Receiver operator curve (ROC) for MACC1 mRNA expression with patient death as binary outcome variable in the Klatskin tumor group. AUC: Area under the curve. n=61.

FIG. 5 Kaplan Meier analysis for overall survival of the entire Klatskin tumor cohort based on MACC1 mRNA expression level with ROC derived cutoff value. The median overall survival time in the Klatskin tumor cohort is 768 days (CI: 255-1251 days). Patients with high MACC1 expression (n=35) have a median overall survival time of 613 days (CI: 300-926); patients with low MACC1 expression (n=26) have a median overall survival time of 2257 days (CI: n/a; p=0.001). (Log rank test)

FIG. 6 MACC1 mRNA expression in Klatskin tumors from patients without tumor recurrence (median 5.25; n=50) and with tumor recurrence (median 14.77; n=24). (Mann Whitney U Test)

FIG. 7 Kaplan Meier analysis for disease free survival of the entire Klatskin tumor cohort based on MACC1 mRNA expression level with ROC derived cutoff value. Patients with high MACC1 expression have a significantly shorter median disease free survival (753 days; CI: 341-1165 days; n=26) than patients with low MACC1 expression (>3119 days; CI: n/a; p<0.001; n=35). (Log rank test)

FIG. 8 Scatter graph: Semi quantitative scoring of immunhistochemical staining (no staining 0, weak staining 1, intermediate staining 2, or strong staining 3) for MACC1 and MACC1 mRNA expression measured by quantitative real time PCR in corresponding tissue samples of Klatskin tumor, intrahepatic cholangiocarcinoma, and hepatocellular carcinoma.

### EXAMPLES

### Material and Methods

### Patients

We obtained tumor specimens of 233 patients undergoing surgery between 1998 and 2003 with HCC (n=77), ICC (n=80), and Klatskin tumors (n=76) from the Department of General-, Visceral-and Transplantation Surgery, Charité Universitätsmedizin Berlin, Campus Virchow Klinikum, Berlin, Germany. Matching peritumoral liver tissue was available in all cases. In addition, we obtained normal bile duct tissue from 3 individuals undergoing surgery for benign liver tumors. Collection of patient tissue and clinical data was approved by the local ethics committee (Charité Universitätsmedizin Berlin), and patients had given informed written consent.

### Tumor Samples

Tissue specimens were collected during surgery, immediately snap frozen in liquid nitrogen and stored at −80° C. and blinded for further analyses.

### Histology and Sample Preparation

Serial sections were made of each tumor specimen for hematoxilin eosin (HE) staining, for microdissection with subsequent RNA isolation and for immunohistochemistry. Of each tumor sample, a section was HE stained following routine protocol. All HE stained sections were given to the Department of Pathology, Charité Universitätsmedizin Berlin, and were examined by a certified pathologist. Tumor diagnosis was confirmed and the respective tumor cell population on the slide was marked as a reference for microdissection. Samples with poor quality (e.g., necrosis) or samples lacking cancerous tissue were omitted from further analyses. With the exception of tumor diagnosis, the pathologist was blinded for all data, including tumor stage and patient outcome.

### RNA Extraction

Tumor samples were microdissected and the material was directly immersed into lysis buffer. Total RNA extraction was performed with the GeneMATRIX Universal RNA Purification Kit (Roboklon GmbH, Germany) according to the manufacturers protocol. Total RNA quality and concentration was assessed with a NanoDrop spectrophotometer and a Bioanalyzer (2100 Bioanalyzer, RNA Pico Chips, Agilent, CA, USA).

qRT-PCR

Real time quantitative two-step RT-PCR (qRT-PCR) was performed using the LightCycler 480 system (Roche Diagnostics). For each sample, a total of 50 ng RNA template was subjected to reverse transcription (RT) (MuLV Reverse Transcriptase, Applied Biosystems), and all subsequent qPCR reactions were done with the same RT reaction. Each qPCR reaction was carried out in duplicate with 5 ng cDNA template in a total reaction volume of 10 μl. For MACC1 and for the house keeping gene glucose-6-phosphate-dehydrogenase (G6PDH), specific hybridization probes were used (synthesis TIB MOLBIOL, Berlin, Germany, and LightCycler hG6PDH Housekeeping Gene Set, Roche Diagnostics) with LightCycler FastStart DNA Master Hyprobe master mix (Roche Diagnostics). The respective primers (synthesis BioTeZ, Berlin, Germany) and hybridization probes used are shown in Table 1.

Quantification was done with the standard curve method. Total RNA of cholangiocarcinoma cell line EGI-1 was diluted to generate a standard curve. For each point on the standard curve, a separate RT reaction with the respective amount of RNA template was carried out. Additionally, a 50 ng RNA template of both cell lines was used as a calibrator, and the target gene expression was calculated as percentage of the respective calibrator sample. Standard curve and calibrator sample, as well as a no-template control were included in every qPCR run.

### Immunohistochemistry

Immunohistologic staining for MACC1 was carried out for 20 specimens of each tumor entity. The samples were chosen based on normalized MACC1 qRT-PCR expression levels, whereas the specimens with the 10 highest and 10 lowest MACC1 expression levels were chosen; for HCC 20 out of 77 samples, for ICC 20 out of 80 samples, and for Klatskin tumors 20 out of 76 samples. Fresh 5 μm cryosections were air dried for one hour at room temperature and then fixed in 10 mM HEPES buffer containing 0.04% glutaraldehyde and 1% glucose. The sections were incubated with 0.9% hydrogen peroxide for 30 minutes, permeabilized with PBS containing 0.5% Triton X100 and 2.5% goat serum for 10 minutes, and treated with biotin blocking reagents (DAKO). After blocking with 5% goat serum for 45 minutes, the sections were incubated with the polyclonal MACC1 antibody (HPA020081, Sigma, 1:250) at 4° C. over night. The sections were incubated with a biotinylated secondary antibody for 30 minutes and treated with a streptavidine peroxidase conjugate for 30 minutes (Strept ABC Complex, DAKO). The slides were developed with DAB for 1 minute (Liquid DAB+Substrate, DAKO) and counterstained with hematoxilin. Sections without primary antibody were used as controls. The slides were examined with an Axioplan 2 microscope (Zeiss) and pictures of representative tumor areas were taken of each specimen at 200 fold magnification (Axiocam HRc camera, Zeiss). The same exposure and whitepoint setting was used for all slides. Pictures were evaluated using the Axovision 4.2 software (Zeiss) and a semi quantitative scoring system was applied. The tumor cells were classified to exhibit no staining (0), weak staining (1), intermediate staining (2), or strong staining (3). Analysis was carried out by two independent investigators who were blinded for the study protocol.

### Statistical Methods

Quantitative variables are expressed as medians. For comparison of groups, Kruskall-Wallys analysis on ranks and Mann-Whitney-U test were used. Inner group comparison was done using the Wilcoxon test.

The main study outcomes were disease free survival and overall patient survival. For each tumor entity, median survival time and 95% confidence interval (CI) was calculated by Kaplan-Meier analysis. MACC1 cut-off values were determined by Receiver-Operating-Characteristics curve analysis (ROC). ROC was carried out for every gene examined with the binary outcome variables for overall survival and disease free survival. The respective expression value yielding the highest Youden index was used as the cut-off value for univariate and multivariate survival analysis.

For the univariate survival analysis, categorical variables were tested using the log-rank test, and metric variables were tested with the Cox proportional hazard model. For the Cox proportional hazards model, the hazard ratio with 95% confidence interval was reported as an estimate of the risk of variable specific death. Variables with P<0.1 in the univariate analysis were included in the multivariate analysis. The multivariate analysis was performed using the backward stepwise procedure for building a Cox proportional hazards model. Time-dependency was tested for every variable in the multivariate analysis and could be excluded. In all statistical tests, P<0.05 was considered statistically significant. Statistical analysis was performed using IBM SPSS Statistics Version 20 (IBM Software Group, USA).

### Results

MACC1 mRNA Expression in Klatskin Tumors and ICC is Significantly Higher than in Corresponding Normal Liver Tissue

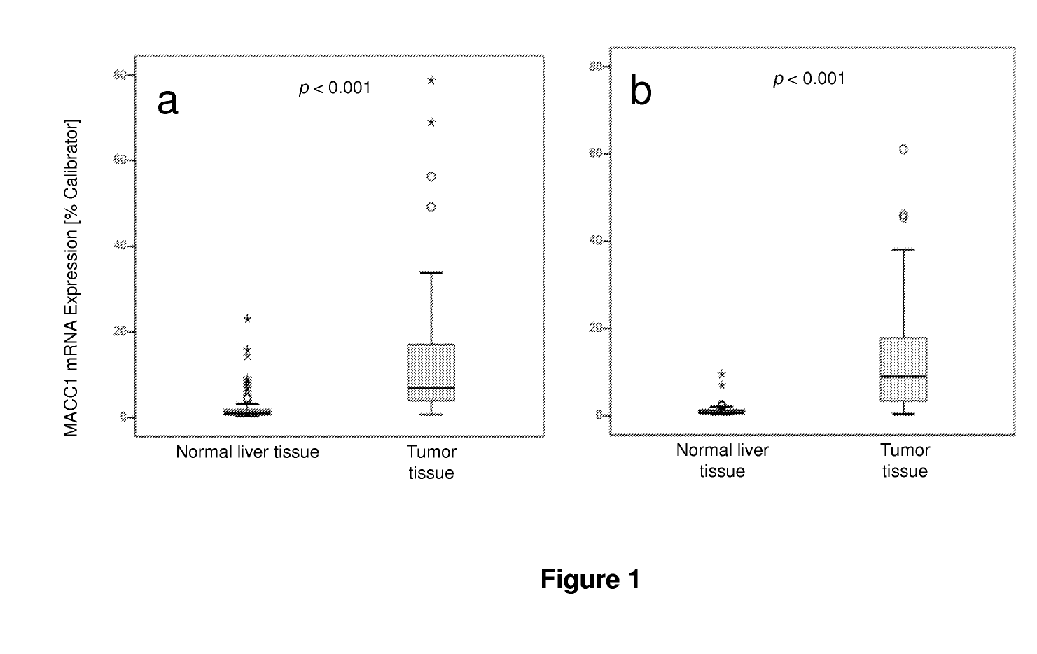

MACC1 mRNA expression could be detected in all tissues examined by qRT-PCR. MACC1 expression was significantly higher in Klatskin tumors compared to corresponding normal liver tissue, with 9.94 vs. 1.06 MACC1 mRNA expression/% Calibrator (p<0.001; FIG. 1A).

MACC1 was also significantly higher expressed in ICC compared to corresponding normal liver tissue, with 8.98 vs. 0.75 MACC1 mRNA expression/% Calibrator (p<0.001; FIG. 1B). The median expression level of MACC1 in biliary tumors was significantly higher than in the bile duct normal tissue controls, which showed MACC1 mRNA expression levels comparable to normal liver tissue.

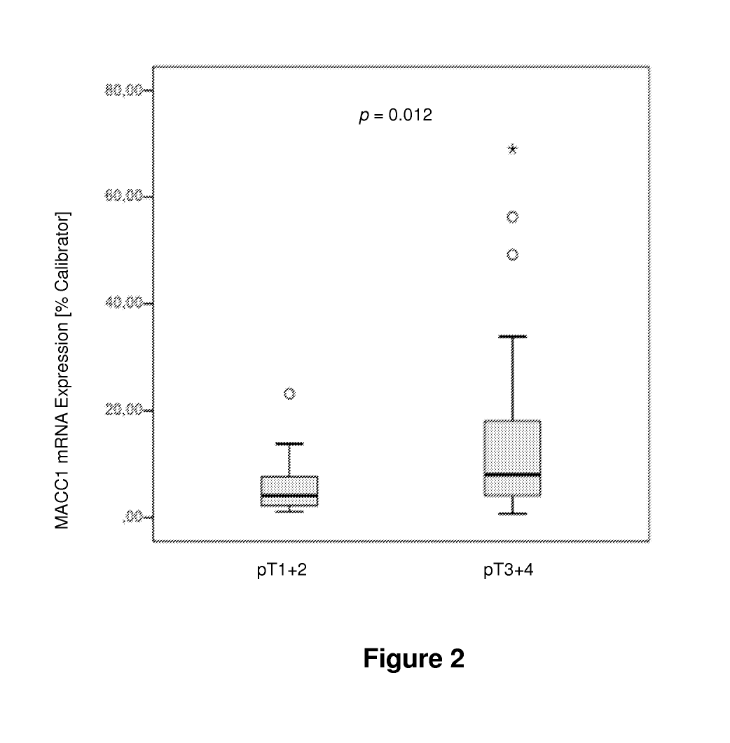

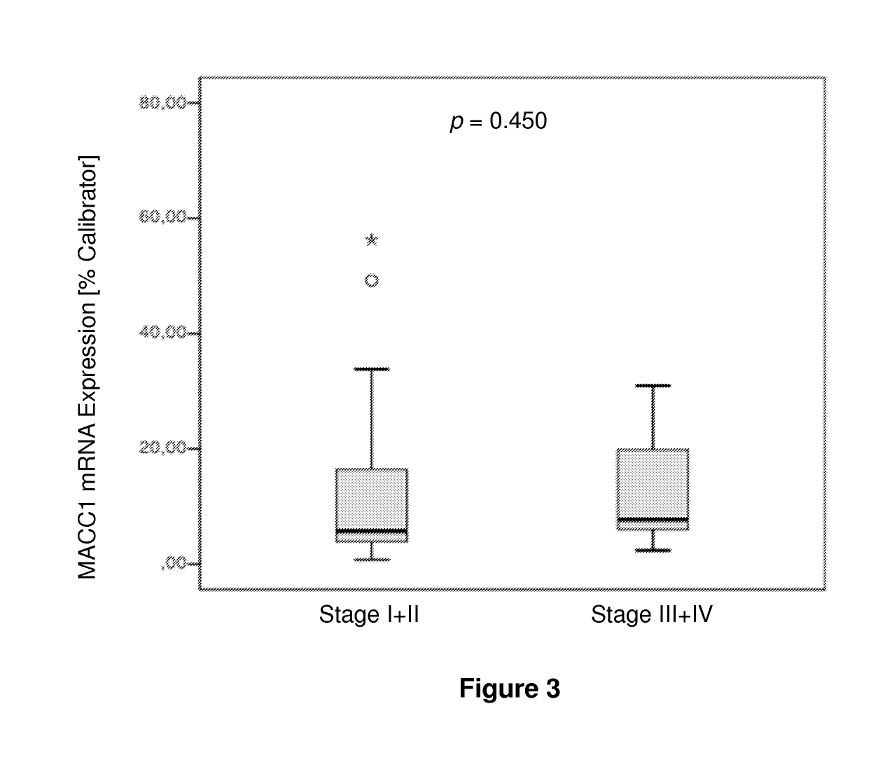

We further investigated the expression levels of MACC1 in selected histopathological subgroups (tumor size pT, lymph node involvement pN, metastasis pM, tumor grading G, and UICC stage) of each tumor entity. In the Klatskin tumor cohort, MACC1 mRNA expression was significantly higher in patients with a tumor size of pT3 and pT4 than with pT1 and pT2, with 4.14 (pT1+2) vs. 8.03 (pT3+4) MACC1 mRNA expression/% calibrator (p=0.012; FIG. 2). MACC1 mRNA expression did neither differ significantly between the different UICC stages (FIG. 3), nor between the other major clinical features (pN, pM, and G).

### Survival Analysis

MACC1 mRNA Expression is a Significant and Independent Prognostic Marker for Overall Survival in Klatskin Tumor Patients

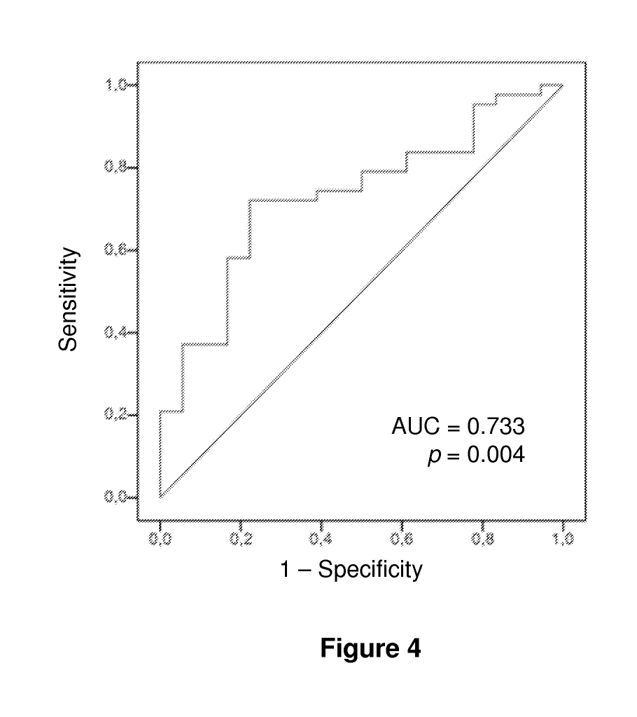

Next, we wished to analyze the prognostic value of MACC1 expression in these hepatobiliary tumor entities. ROC analysis was used to determine a cutoff value for overall survival and disease free survival. A total number of 61 patients out of 76 patients in the Klatskin tumor cohort survived more than 30 days after surgery, had complete clinical follow up and were considered for survival analysis. In this group, ROC analysis for overall survival and disease free survival both yielded a significant area under the curve (AUC) for MACC1 mRNA expression. Thus, the Klatskin tumor cohort was divided in low and high MACC1 expressors according to the cutoff value calculated by Youden index, which is 8.96 MACC1 mRNA expression/% calibrator (sensitivity 70.8%; specificity 72%) (FIG. 4). Epidemiological, clinical, and histopathological data of Klatskin tumor patients grouped according to MACC1 mRNA expression status are reported in Table 2.

### Univariate Survival Analysis

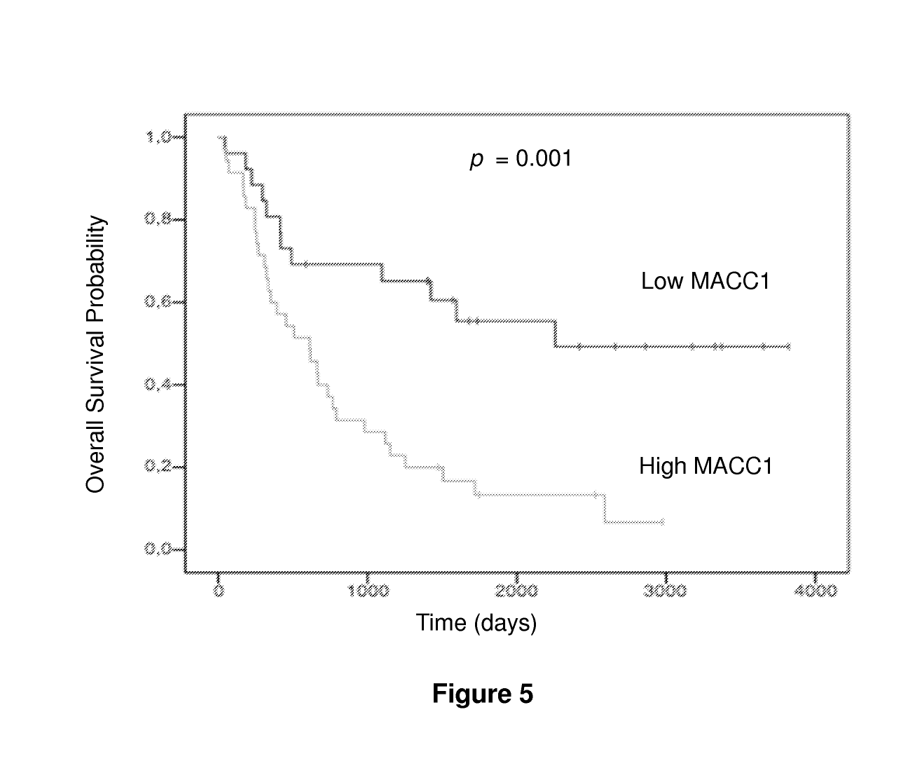

The median overall survival time in the Klatskin tumor cohort was 768 days (CI: 255-1251 days). Patients with high MACC1 expression had a median overall survival time of 613 days (CI: 300-926), which was significantly shorter than patients with low MACC1 expression (median survival time: 2257 days; CI: n/a; p=0.001) (FIG. 5). Survival analysis was repeated using the COX regression model with MACC1 as a binary as well as continuous variable. Both regression models confirmed the results of the log rank test. With MACC1 as continuous variable, the HR was 1.047 (CI: 1.024-1.072; p>0.001).

Other variables we found to have a significant influence on overall survival were lymph node status pN (pN0: 1505 days (CI: 870-2140 days); pN1: 418 days (CI: 224-612 days); p=0.011). Tumor size pT (p=0.07), occurrence of metastasis M (p=0.209), tumor differentiation G (p=0.368), and resection margin R (p=0.169) were not significant parameters in our study. Patient age at surgery (p=0.545) and gender (p=0.950) had no significant effect on overall survival in the Klatskin tumor cohort.

### Multivariate Survival Analysis

We conducted multivariate analysis to evaluate whether MACC1 mRNA expression is an independent factor for overall survival in the Klatskin tumor cohort. We used the COX regression model with the stepwise backwards procedure with all variables that had a significance of P<0.1 in the univariate analysis. Independent prognostic significance was detected for MACC1 expression with the ROC derived cutoff (HR 2.777; CI: 1.389-5.555; P=0.004) and lymph node status pN (HR 2.114; CI: 1.114-4.015; p=0.022). Therefore, we found MACC1 mRNA expression to be a strong and independent predictor of overall survival in Klatskin tumor patients.

MACC1 mRNA Expression is a Significant Prognostic Marker for Disease Free Survival in Klatskin Tumor Patients

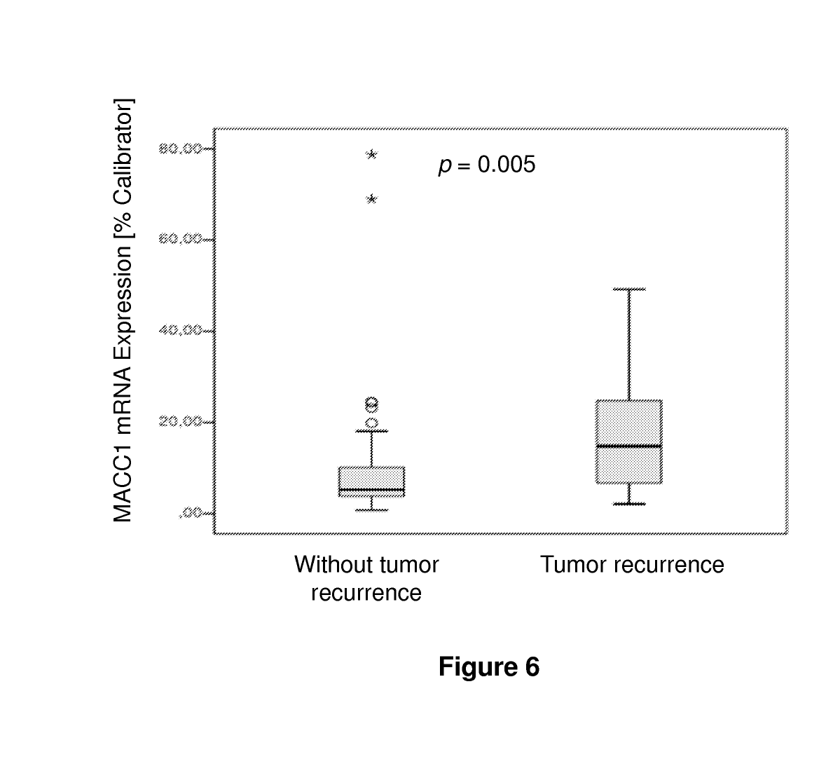

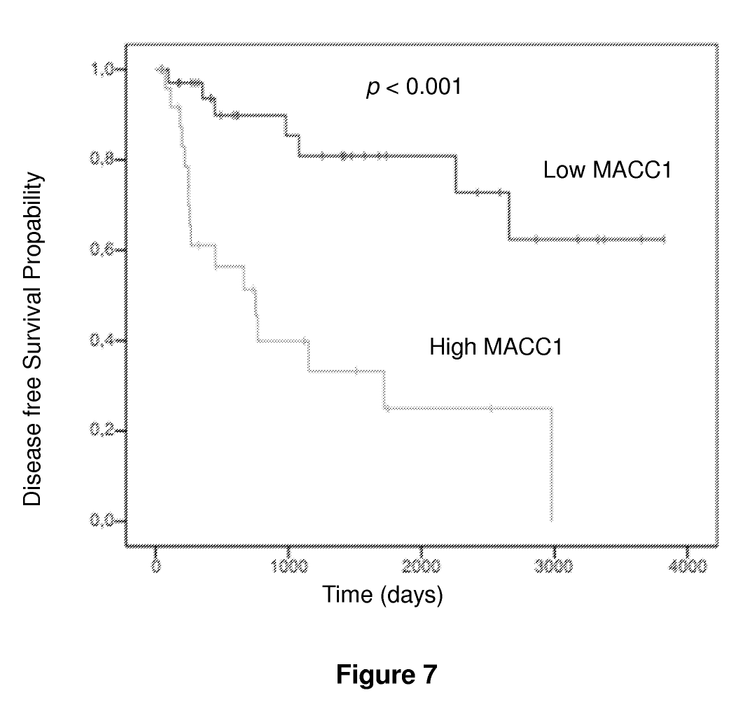

Patients with a history of tumor recurrence had significantly higher MACC1 mRNA expression than patients without tumor recurrence (FIG. 6). Moreover, patients with high MACC1 expression had a significantly shorter median disease free survival (753 days; CI: 341-1165 days) than patients with low MACC1 expression (>3119 days; CI: n/a; p<0.001) (FIG. 7).

Validation of Results Obtained by qRT-PCR with Immunohistochemistry

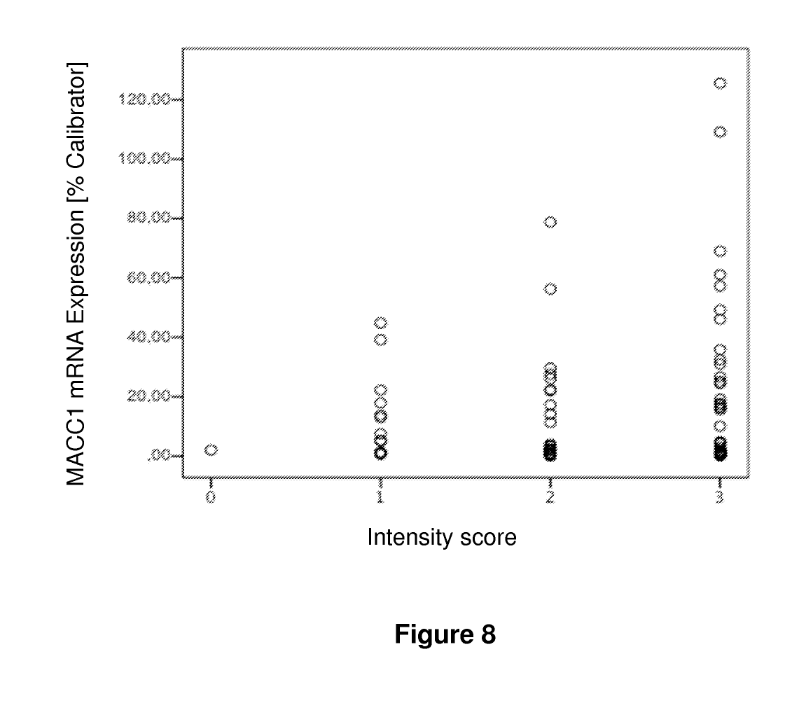

To validate the results obtained by qRT-PCR on the protein level, tissue samples were stained for MACC1. The results of the semi quantitative scoring of the staining intensity were plotted against the quantitative expression values measured by qRT-PCR. A representative scattergraph of the MACC1 expression values is shown in FIG. 8. Given the lack of precise quantitation using immunohistochemistry, we did not perform survival analysis using these results.

In summary, here we report a first study measuring the expression for MACC1 mRNA quantitatively in microdissected hepatobiliary tumors. In our study, qRT-PCR was a suitable method to determine MACC1 expression in frozen tumor tissue. Most importantly, we identified MACC1 as a significant and independent prognostic biomarker for overall survival and disease free survival in Klatskin tumor patients.

### CONCLUSION

In summary, our study identifies MACC1 as a highly prognostic biomarker for overall survival and disease free survival in Klatskin tumor patients. Prospective validation of MACC1 mRNA expression in Klatskin tumors may eventually make it a tool in clinical decision making to allocate patients suitable for curative surgery, which includes liver transplantation.

TABLE 1
Primers and probes for quantitative
real time PCR

| Gene | | Sequence | SEQ ID |
|---|---|---|---|
| MACC1 | Forward | TTC TTT TGA TTC CTC | NO. 3 |
| | primer | CGG TGA | |
| | Reverse | ACT CTG ATG GGC ATG | NO. 4 |
| | primer | TGC TG | |
| | FITC | GCA GAC TTC CTC AAG AAA | NO. 5 |
| | probe | TTC TGG AAG ATC TA | |
| | Red640 | AGT GTT TCA GAA CTT CTG | NO. 6 |
| | probe | GAC ATT TTA GAC GA | |

| | |
|---|---|
| G6PDH | LightCycler hG6PDH Housekeeping Gene Set |
| | (Cat. No. 03261883001, Roche Diagnostics) |

TABLE 2
Clinical features of the Klatskin Series (n = 61) according to ROC
based MACC1 grouping considered for survival analysis.

| | | Total | MACC1 | | |
|---|---|---|---|---|---|
| | | number | low | high | |
| Clinical features | | of Cases | (n = 26) | (n = 35) | P† |
| Status | | | | | <0.001 |
| | alive | 18 | 14 (53.8) | 4 (11.4) | |
| | dead | 43 | 12 (46.2) | 31 (88.6) | |
| Gender | | | | | 0.395 |
| | male | 40 | 14 (53.8) | 15 (42.9) | |
| | female | 36 | 12 (46.2) | 20 (57.1) | |
| Age | | 61 | 60 ± 10 | 60 ± 10 | 0.830 |
| Size | | | | | 0.744 |
| | pT1 | 2 | 1 (3.85) | 1 (3.03) | |
| | pT2 | 11 | 6 (23.07) | 5 (15.15) | |
| | pT3 | 43 | 18 (69.23) | 25 (75.75) | |
| | pT4 | 3 | 1 (3.85) | 2 (6.07) | |
| Node Involvement | | | | | 0.239 |
| | pN0 | 33 | 17 (65.4) | 16 (50) | |
| | pN1 | 25 | 9 (34.6) | 16 (50) | |
| Metastasis | | | | | 0.202 |
| | pM0 | 57 | 26 (100) | 31 (93.3) | |
| | pM1 | 2 | 0 (0) | 2 (6.7) | |
| Histological Grade | | | | | 0.039 |
| | G1 | 2 | 1 (5.6) | 1 (3.7) | |
| | G2 | 35 | 17 (94.4) | 18 (66.7) | |
| | G3 | 8 | 0 (0) | 8 (29.6) | |
| Resection Margin | | | | | 0.806 |
| | R0 | 38 | 17 (94.4) | 21 (67.7) | |
| | R1 | 17 | 7 (5.6) | 10 (32.3) | |

Table values are given as mean ± SD for continuous variables and n (%) for categorical variables.
†P value is for t test (continuous variable) or χ² test (categorical variables).

1. A method of diagnosis and prognosis of a hepatobiliary tumor, the method comprising the step of determining expression of MACC1 polypeptide or of a nucleic acid encoding said MACC1 polypeptide in a biological sample, wherein said MACC1 polypeptide has at least 80% identity to the amino acid sequence with SEQ ID NO. 1.

2. The method of claim 1, wherein the method is performed in vitro.

3. The method of claim 1, wherein the hepatobiliary tumor is a cholangiocarcinoma or a gall bladder carcinoma, preferably the hepatobiliary tumor is a cholangiocarcinoma, more preferably the hepatobiliary tumor is a Klatskin tumor.

4. The method of claim 1, wherein the expression level of the MACC1 polypeptide or the nucleic acid encoding said MACC1 polypeptide is elevated if expression in the biological sample exceeds expression in a disease-free comparative sample and/or if expression in the biological sample exceeds a predetermined threshold value.

5. The method of claim 1, wherein said method comprises the steps of:
determining the expression level of MACC1 polypeptide or of a nucleic acid encoding said MACC1 polypeptide in a biological sample;
determining the expression level of MACC1 polypeptide or of a nucleic acid encoding said MACC1 polypeptide in a comparative sample; and
comparing the expression level of the biological sample with the expression level of the comparative sample.

6. The method of claim 1, wherein said method comprises the steps of:
determining the expression level of MACC1 polypeptide or of a nucleic acid encoding said MACC1 polypeptide in a biological sample; and
comparing the expression level of the biological sample with a predetermined threshold value.

7. The method of claim 1, wherein the biological sample is derived from a subject to be investigated.

8. The method of claim 1, wherein the biological sample is a tissue biopsy, preferably a tissue biopsy comprising hepatic, bilial and/or putative tumorous tissue.

9. The method of claim 1, wherein expression is determined using a polypeptide which binds specifically to the MACC1 polypeptide, wherein said MACC1 polypeptide has at least 80% identity to the amino acid sequence with SEQ ID NO. 1.

10. The method of claim 9, wherein the polypeptide is an antibody.

11. The method of claim 1, wherein expression is determined using a nucleic acid molecule that hybridizes under stringent conditions to a nucleic acid sequence encoding MACC1 polypeptide or to the reverse complement thereof, wherein said MACC1 polypeptide has at least 80% identity to the amino acid sequence with SEQ ID NO. 1

12. The method of claim 11, wherein the nucleic acid molecule hybridizes under stringent conditions to a nucleic acid sequence with SEQ ID NO. 2 or to a reverse complement thereof.

13. The method of claim 1, wherein expression of MACC1 polypeptide or of a nucleic acid encoding said MACC1 polypeptide is determined by hybridisation, a PCR, a quantitative or semi-quantitative RT-PCR, a “real-time” (RT)-PCR, antigen-antibody binding, an ELISA, an optical proteome analysis, a one- or multi-dimensional gel electrophoresis, an analysis by mass spectrometry, a chromatography, a sequencing procedure, a methylation analysis, a SNP-determination or by a combination including one or more of these methods.

14. An isolated nucleic acid molecule that hybridizes under stringent conditions to a nucleic acid sequence encoding MACC1 polypeptide or to the reverse complement thereof, wherein said MACC1 polypeptide has at least 80% identity to the amino acid sequence with SEQ ID NO. 1, for use in a method of diagnosis of a hepatobiliary tumor according to claim 1, wherein said method comprises the step of isolating the biological sample from a subject.

15. The isolated nucleic acid molecule of claim 14, wherein the nucleic acid molecule hybridizes under stringent conditions to a nucleic acid sequence with SEQ ID NO. 2 or to a reverse complement thereof.

16. An isolated polypeptide which binds specifically to the MACC1 polypeptide, wherein said MACC1 polypeptide has at least 80% identity to the amino acid sequence with SEQ ID NO. 1, for use in a method of diagnosis and prognosis of a hepatobiliary tumor, wherein said method comprises the step of isolating a biological sample from a subject.

17. The isolated polypeptide of claim 16, wherein said isolated polypeptide is an antibody.

18. A method of diagnosis and prognosis of hepatobiliary tumor, wherein MACC1 polypeptide or of a nucleic acid encoding said MACC1 polypeptide, wherein said MACC1 polypeptide has at least 80% identity to the amino acid sequence with SEQ ID NO. 1, is used as a biomarker.

19. A method for evaluating effectiveness of a treatment of hepatobiliary tumors, wherein the steps of a method of claim 1 are used.

20. A method for identifying a candidate compound useful in treatment of hepatobiliary tumors, comprising the steps of:
contacting a mammal subject presenting a hepatobiliary tumor with a candidate compound:
determining the expression level of MACC1 polypeptide or of a nucleic acid encoding said MACC1 polypeptide in a biological sample isolated from said subject;
wherein a decrease in the expression level indicates that the candidate compound is useful in treatment of hepatobiliary tumors.
